(19) United States
(12) Patent Application Publication
(10) Pub. No.: US 2021/0228384 A1
(43) Pub. Date: Jul. 29, 2021

(54) LIGHTWEIGHT PROSTHETIC FOOT

(71) Applicant: HARIPRIYA SUMANA GOSAKAN, BANGALORE (IN)

(72) Inventor: HARIPRIYA SUMANA GOSAKAN, BANGALORE (IN)

(21) Appl. No.: 17/259,545

(22) PCT Filed: Jul. 12, 2019
(86) PCT No.: PCT/IN2019/050517

§ 371 Date: Jan. 11, 2021

(30) Foreign Application Priority Data
Jul. 13, 2018 (IN) 201841026267

(51) Int. Cl. A61F 2/66

(52) U.S. Cl. CPC A61F 2/66; A61F 2002/5053; A61F 2002/6664; A61F 2002/6614

(57) ABSTRACT

A prosthetic foot (200) that does not require metallic connectors to attach carbon fiber leaf springs (201) and (202) is provided. The carbon fiber leaf springs (201) and (202) are attached together by three-dimensionally stitching prepreg preforms of the carbon fiber leaf springs (201) and (202) at the junction (203) of the carbon fiber leaf springs (201) and (202), and then further curing the stitch attached prepreg preforms. In another embodiment, a woven interlayer attached the two carbon fiber leaf springs (201) and (202).

### CROSS-REFERENCE TO RELATED APPLICATIONS

This PCT application claims priority to and the benefit of provisional patent application titled “Lightweight Prosthetic Foot”, application number 201841026267, filed in the Indian Patent Office on 13 Jul. 2018. The specification of the above referenced patent application is incorporated herein by reference in its entirety.

### BACKGROUND

The apparatus disclosed herein, in general, relates to a medical device, and in particular, relates to a prosthetic foot.

Amputees with a loss of a limb experience discomfort while using heavy prosthetic feet. FIG. 1 (prior art) exemplarily illustrates a prosthetic foot 100 with a heavy metallic connector 103 connecting two leaf springs 101 and 102. There is an unmet need for eliminating metallic connectors and fittings in prosthetic feet. The metallic connector between leafsprings in a composite foot may weigh as much as between 30 gm to 70 gm. A leafspring herein refers to a composite multilayer laminate that flexes and provides energy return in a prosthetic foot. The cavity to house the bolt of the metallic connector weakens the carbon fiber leafspring structure in the prior art. Hence, to compensate for this loss in strength, additional composite material of approximately 70 to 100 gm will need to be added to the leaf springs. In addition, the cavity and joint area in proximity to the metallic connector is highly stressed and is potentially an area of crack initiation.

### SUMMARY OF THE INVENTION

The apparatus disclosed herein addresses the above recited unmet need for eliminating or reducing the number of metallic connectors and fittings in prosthetic feet.

Elimination of metallic connectors between leafsprings can potentially result in significant weight savings, reducing the potential of crack propagation and improving wear and comfort for the amputee through weight reduction.

### BRIEF DESCRIPTION OF THE DRAWINGS

FIG. 1 (prior art) exemplarily illustrates a prosthetic foot with a heavy metallic connector to connect composite leafsprings.

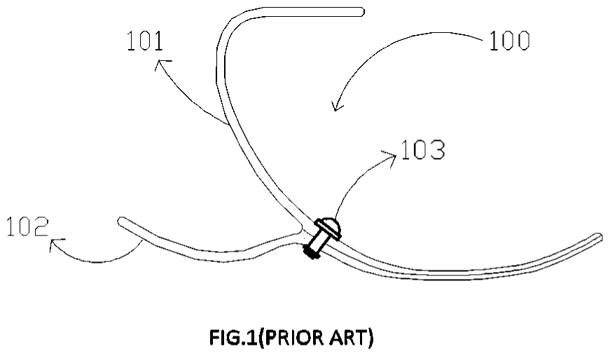

FIG. 2 illustrates a prosthetic foot without the need for a metallic connector to connect composite leafsprings.

FIGS. 3A and 3B illustrates the stitch orientation between carbon fiber leafsprings.

FIG. 4A illustrates the details of the woven interlayer between carbon fiber leafsprings.

FIG. 4B illustrates the assembly of the woven interlayer between carbon fiber leafsprings.

### DETAILED DESCRIPTION OF THE INVENTION

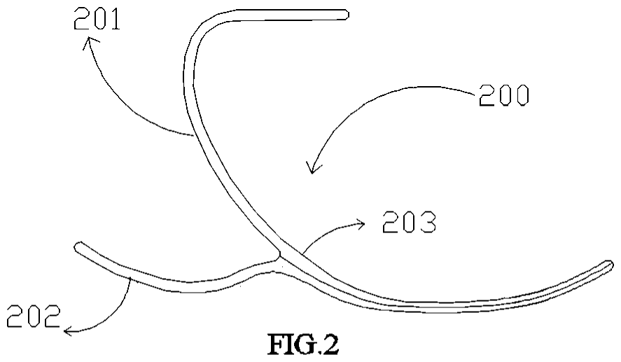

FIG. 2 illustrates a prosthetic foot 200 without the need for a metallic connector 103. In the prosthetic foot 200 disclosed herein, the composite leaf springs 201 and 202 are attached together by three-dimensionally stitching prepreg preforms of the composite leaf springs 201 and 202 at the junction 203, and then further curing the stitch attached prepreg preforms. Three-dimensional stitching eliminates the possibility of delamination between the two layers of the carbon fiber leaf springs 201 and 202.

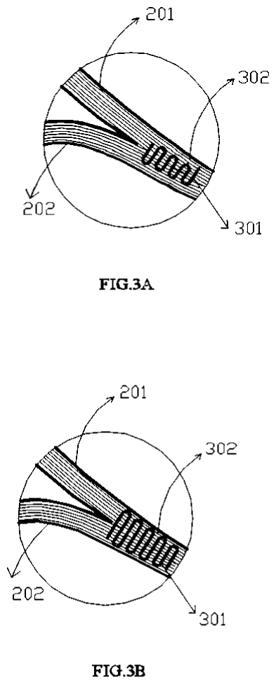

FIG. 3A, 3B illustrates the stitch orientation between carbon fiber leafsprings 201 and 202. After the stitch attachment of the prepeg layers, a superficial prepeg layer 302 may be overlaid over the stitch attached layers to avoid the visual exposure of the stitches 301. In FIG. 3B the depth of the stitch is deeper than in FIG. 3A.

Different types of stitches, such as a running stitch, lockstitch, chain stitch, or other stitches may be incorporated using a stitching machine.

The thread for stitching is preferably a material which can take a sharp bend radius, for example Aramid fiber. Optionally carbon or glass fibers may also be used.

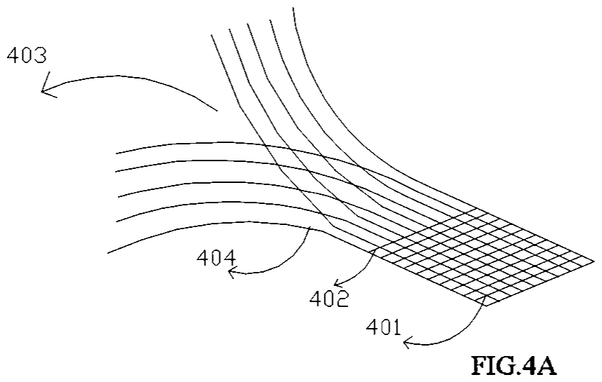

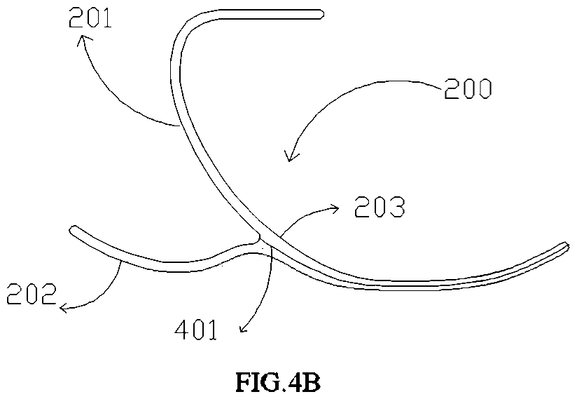

FIG. 4A illustrates the details of the woven interlayer between carbon fiber leafsprings 201 and 202. FIG. 4B illustrates the assembly of the woven interlayer between carbon fiber leafsprings 201 and 202. The three dimensional attachment between the carbon fiber leafsprings 201 and 202 may also be executed by using a woven matt 401. The Y oriented fibers 402 in the woven matt is removed in the area 403 where leaf springs 201 and 202 depart. In the departing area 403, some of the X oriented fibers 404 are taken to adhere to top leaf spring 201 , and the rest of the X oriented fibers are taken to adhere to the lower leaf spring 202. As the junction area maintains the woven structure, the X oriented fibers 404 will not separate while the leaf springs 201 and 202 are pulled apart. Therefore, an effective composite connection will be established at the junction eliminating the risk of delamination between the two leaf springs 201 and 202.

Consider a prosthetic foot 200 comprising a first composite leafspring 201 and a second composite leafspring 202. An woven interlayer 301 exists between the first composite leafspring 201 and second composite leafspring 202, wherein a portion of the fibers from the woven layer in the direction along the length of the foot is inserted into the first composite leafspring 201, and the rest of the fibers in the direction along the length of the foot is inserted into the second leafspring 202. In this case, the method of manufacturing a prosthetic foot comprises laying a first prepeg layer along a first part of a mold, laying a second prepeg surface on a second part of the mold, laying an woven interlayer 401 prepeg between the first composite leafspring 201 and second composite leafspring 202. A portion of the fibers from the woven interlayer 401 prepeg in the direction along the length of the foot is inserted into the first composite leafspring 201, and the rest of the fibers in the direction along the length of the foot is inserted into the second leafspring 202. The mold is vacuum bagged, pressurized and high temperature cured.

The prosthetic foot 200 is manufactured using one or a combination of the following materials: carbon fiber composites, glass fiber composites, aramid fiber composites, plastics, metal, etc. For example, the prosthetic foot 200 comprises carbon fiber in a thermosetting or thermoplastic matrix.

For example, unidirectional carbon fiber prepegs may be laid in a multiaxial layup sequence to form a first and a second leaf spring. The two leaf springs are stitched using Aramid fiber of 2000 denier and high temperature cured in a pressurized vacuum bagged environment in a metallic mold at a high temperature of 150 Celcius.

For example, each carbon fiber leafspring may be of thickness 3mm to 9mm. The fiber layup sequence may comprise the following: Top woven 0/90, +45, −45, 0, 0, 0, 0, 0, 0, 0, 0−45,+45, Lower woven 0/90. The thickness of each prepeg layer may be between 0.1 mm to 0.25 mm. To increase thickness, the number of prepeg layers may be increased.

Optionally a polyurethane insert may be introduced in the area between the carbon fiber leafsprings for an additional cushioning effect in the transition phase.

The leaf springs 201 and 202 are made of carbon fiber composites, glass fiber composites, aramid fiber composites or a combination of different types of fiber composites in a plastic matrix. The plastic matrix may be a thermosetting or thermoplastic matrix. Example of thermosetting matrix includes epoxy, vinyl ester, polyester, polyurethane etc. Examples of thermoplastic matrix include high density polyethylene, PEEK etc.

The three dimensional attachment between leaf springs may be between any two or more leaf springs in proximity.

The foregoing examples have been provided merely for explanation and are in no way to be construed as limiting of the prosthetic foot 200 disclosed herein. While the prosthetic foot 200 has been described with reference to various embodiments, it is understood that the words, which have been used herein, are words of description and illustration, rather than words of limitation. Furthermore, although the prosthetic foot 200 has been described herein with reference to particular means, materials, and embodiments, the prosthetic foot 200 is not intended to be limited to the particulars disclosed herein; rather, the prosthetic foot 200 extends to all functionally equivalent structures, methods and uses, such as are within the scope of the appended claims. While multiple embodiments are disclosed, it will be understood by those skilled in the art, having the benefit of the teachings of this specification, that the prosthetic foot 200 disclosed herein is capable of modifications and other embodiments may be effected and changes may be made thereto, without departing from the scope and spirit of the prosthetic foot 200 disclosed herein.

1. A prosthetic foot comprising:
a first composite leafspring;
a second composite leafspring; and
a three dimensional composite fiber stitch between said first leafspring and second leafspring.

2. The prosthetic foot of claim 1, wherein said composite leafspring further comprises a composite fiber in a resin matrix.

3. The prosthetic foot of claim 2, wherein said composite fiber is one of or a combination of carbon fiber, aramid fiber and glass fiber.

4. The prosthetic foot of claim 2, wherein said resin matrix is one of epoxy, vinyl ester, polyester or polyurethane.

5. The prosthetic foot of claim 1, wherein said composite fiber stitch comprises a stitch that is composed of one or a combination of aramid, carbon or glass fibers.

6. A prosthetic foot comprising:
a first composite leafspring;
a second composite leafspring; and
an woven interlayer between said first composite leafspring and second composite leafspring, wherein a portion of the fibers from the woven layer in the direction along the length of the foot is inserted into the first composite leafspring, and the rest of the fibers in the direction along the length of the foot is inserted into the second leafspring.

7. A method of manufacturing a prosthetic foot comprising:
stitching a first and a second prepeg layer using a composite fiber;
laying the first prepeg layer along a first part of a mold;
laying the second prepeg surface on a second part of the mold;
wherein the junction between said first prepeg layer and second prepeg layer is stitch attached; and
pressurizing and curing said first prepeg layer and second prepeg layers.

8. (canceled)
